(19) United States
(12) Patent Application Publication
(10) Pub. No.: US 2006/0084806 A1
(43) Pub. Date: Apr. 20, 2006

(54) Processes for the preparation of imidazo[1,2-a] pyridine derivatives

(76) Inventors: Ramasubramanian Sridharan, Kopar Khairane (IN); Shekhar Bhaskar Bhirud, Vashi (IN); Vandanapu Loka Appala Purushotham, Kopar Khairane (IN)

Correspondence Address: M. CARMEN & ASSOCIATES, PLLC, 170 OLD COUNTRY ROAD, SUITE 400, MINEOLA, NY, 11501, US

(21) Appl. No.: 11/186,662

(22) Filed: Jul. 21, 2005

Related U.S. Application Data
(60) Provisional application No. 60/589,836, filed on Jul. 21, 2004.

(51) Int. Cl. C07D 471/02

(52) U.S. Cl. 546/121

(57) ABSTRACT

A process for the preparation of imidazo[1,2-a]pyridine derivatives of the formula I: 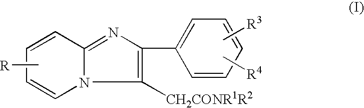
wherein R is hydrogen, halogen or a C₁-C₄ alkyl group; R¹ and R² are independently hydrogen, a straight or branched C₁-C₆ alkyl group which is unsubstituted or substituted by one or more halogen atoms, hydroxyl, N(C₁-C₄ alkyl)₂, carbamoyl or C₁-C₄ alkoxy radicals, a C₁-C₆ alkyl hydroxy group, a C₃-C₆ cycloalkyl radical, a benzyl radical, a phenyl radical or R¹ and R² together with the nitrogen atom to which they are bonded are joined together to form a substituted or unsubstituted heterocyclic group optionally containing one or more additional heterocyclic atoms; and R³ and R⁴ are independently hydrogen, halogen or a C₁-C₄ alkyl group, or a pharmaceutically acceptable salt thereof, the process comprising (a) reacting an imidazo[1,2-a]pyridine carboxylic acid of the formula II 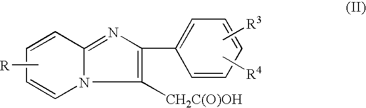
wherein R, R³ and R⁴ have the aforestated meanings with a halogenating agent in the absence of a solvent to form an acid halide intermediate and (b) reacting the acid halide intermediate with an amine of the formula HNR¹R² wherein R¹ and R² have the aforestated meanings to form the compound of formula I.

### PRIORITY

This application claims the benefit under 35 U.S.C. §119 to Provisional Application No. 60/589,836, filed Jul. 21, 2004 and entitled “PROCESSES FOR THE PREPARATION OF ZOLPIDEM TARTARATE”, the contents of which are incorporated by reference herein.

### BACKGROUND OF THE INVENTION

1. Technical Field

The present invention generally relates to an improved process for the preparation of imidazo[1,2-a]pyridine derivatives.

2. Description of the Related Art

The present invention is directed to an improved process for the preparation of imidazo[1,2-a]pyridine derivatives such as zolpidem (also known as N,N-dimethyl-2-[6-methyl-2-(4-methylphenyl)imidazo[1,2-a]pyridine-3-yl]acetamide) and having the formula:

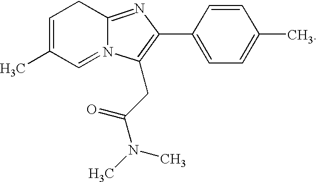

Zolpidem is a hypnotic agent, classified as a non-benzodiazepine hypnotic of the imidazopyridine class. Zolpidem tartrate (2:1) is used in the treatment of short-term insomnia and marketed under the brand name Ambien®.

U.S. Pat. No. 4,382,938 discloses imidazo[1,2-a]pyridine derivatives such as zolpidem and processes for their preparation. In one process, the chloride of zolpidic acid (also known as 2-(4-chlorophenyl)imidazo[1,2-a]pyridine-3-acetic acid) is added to a suspension of N,N-dimethyl-chloro-methyleneiminium chloride, which is prepared by adding oxalyl chloride to dimethylformamide (DMF). An amine in dry dimethylformamide is then added to the suspension to prepare zolpidem.

The major disadvantages of the prior art includes the use of oxalyl chloride as a chlorinating agent in a solvent, which is expensive, and also results in inconvenient processes.

Accordingly, there remains a need for an improved process for the preparation of imidazo[1,2-a]pyridine derivatives such as zolpidem that eliminates and reduces the problems of the prior art on a commercial scale in a convenient and cost efficient manner.

### SUMMARY OF THE INVENTION

In accordance with one embodiment of the present invention, a process for the preparation of imidazo[1,2-a]pyridine derivatives of the formula I is provided: 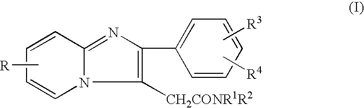
wherein R is hydrogen, halogen or a C₁-C₄ alkyl group; R¹ and R² are independently hydrogen, a straight or branched C₁-C₆ alkyl group which is unsubstituted or substituted by one or more halogen atoms, hydroxyl groups, N(C₁-C₄ alkyl)₂, carbamoyl or C₁-C₄ alkoxy radicals, a C₃-C₆ cycloalkyl radical, a benzyl radical, a phenyl radical or R¹ and R² together with the nitrogen atom to which they are bonded are joined together to form a substituted or unsubstituted heterocyclic group optionally containing one or more additional heterocyclic atoms; and R³ and R⁴ are independently hydrogen, halogen or a C₁-C₄ alkyl group, or a pharmaceutically acceptable salt thereof, the process comprising (a) reacting an imidazo[1,2-a]pyridine carboxylic acid of the formula II 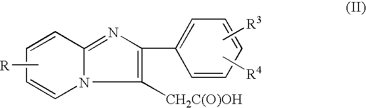
wherein R, R³ and R⁴ have the aforestated meanings with a halogenating agent in the absence of a solvent to form an acid halide intermediate and (b) reacting the acid halide intermediate with an amine of the formula HNR¹R² wherein R¹ and R² have the aforestated meanings to form the compound of formula I.

In accordance with a second embodiment of the present invention, a process for preparing zolpidem is provided comprising the steps of (a) reacting zolpidic acid with a halogenating agent in the absence of a solvent to form a zolpidic acid halide intermediate and (b) reacting the zolpidic acid halide intermediate with dimethylamine to form zolpidem.

In accordance with a third embodiment of the present invention, a process for preparing zolpidem is provided comprising the steps of (a) reacting zolpidic acid with a halogenating agent in the absence of a solvent to form a zolpidic acid halide intermediate and (b) reacting the zolpidic acid halide intermediate with an aqueous solution of dimethylamine to form zolpidem.

In accordance with a fourth embodiment of the present invention, a process for preparing zolpidem tartrate salt is provided comprising the steps of (a) forming a zolpidic acid halide intermediate and from zolpidic acid in the absence of a solvent (b) reacting the zolpidic acid halide intermediate with dimethylamine to form zolpidem base; and (c) forming zolpidem tartrate salt from the zolpidem base.

Advantages of the present invention include at least:

1. A simple process for preparing zolpidem including reducing the number of reaction materials needed to produce zolpidem compared to the prior art.

2. Avoids the use of dimethylamine gas, which requires special equipment.

3. Easier and more economical production on a commercial scale because the reaction conditions are simple, avoids low temperatures, and uses safe and inexpensive reactants and techniques.

4. Neat halogenating agent is used in preparing the zolpidic acid halide intermediate thereby avoiding the use of to provide a more environmentally friendly process than the prior art.

5. Higher yields and purity.

### DETAILED DESCRIPTION OF THE PREFERRED EMBODIMENTS

The present invention involves a process for the preparation of imidazo[1,2-a]pyridine derivatives of formula I: 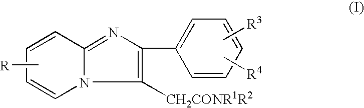
wherein R is hydrogen, halogen or a C₁-C₄ alkyl group; R¹ and R² are independently hydrogen, a straight or branched C₁-C₆ alkyl group which is unsubstituted or substituted by one or more halogen atoms, hydroxyl groups, N(C₁-C₄ alkyl)₂, carbamoyl or C₁-C₄ alkoxy radicals, a C₃-C₆ cycloalkyl radical, a benzyl radical, a phenyl radical or R¹ and R² together with the nitrogen atom to which they are bonded are joined together to form a substituted or unsubstituted heterocyclic group optionally containing one or more additional heterocyclic atoms, e.g., O, S or N; and R³ and R⁴ are independently hydrogen, halogen or a C₁-C₄ alkyl group, or pharmaceutically acceptable salts thereof. Preferably, only one of R¹ and R² are hydrogen. Representative examples of R¹ and R² are independently methyl, ethyl, propyl, isopropyl, butyl, isobutyl, pentyl, hexyl, cyclohexyl, heptyl, octyl, 2-ethylhexyl, nonyl, decyl, dodecyl, stearyl, oleyl, phenyl, benzyl, and the like, containing, e.g., up to about 20 carbon atoms, preferably no more than about 18 carbon atoms and more preferably no more than about 12 carbon atoms. Representative groups in the case where R¹ and R² together with the nitrogen atom to which they are bonded are joined together to form a heterocyclic compound include substituted or unsubstituted cyclic amines such as pyrrolidines, piperidines, piperazines, morpholines, and the like. R¹ and R² can also independently be alkyl groups substituted with one or more heterocyclic substituents.

Generally, one embodiment of the process of the present invention involves (a) reacting an imidazo[1,2-a]pyridine carboxylic acid of the formula II 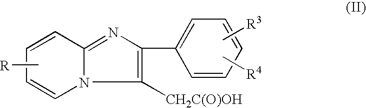
wherein R, R³ and R⁴ have the aforestated meanings with a halogenating agent in the absence of a solvent to form an acid halide intermediate and (b) reacting the acid halide intermediate with an amine of the formula HNR¹R² wherein R¹ and R² have the aforestated meanings to form the compound of formula I. Representative examples of the starting compound of formula II are known. See, e.g., U.S. Pat. No. 4,382,938, the contents of which are incorporated by reference herein.

The halogenating agents for use in step (a) of the process can be any suitable chlorinating agent. Useful chlorinating agents include, but are not limited to, phosphorous oxychloride, phosphorous trichloride, phosphorous pentachloride, thionyl chloride and the like. The halogenting agent may be used in a ratio of about 1:2 w/v to about 1:3 w/v ratio with respect to the imidazo[1,2-a]pyridine carboxylic acid such as zolpidic acid (imidazo[1,2-a]pyridine carboxylic acid:phosphorous oxychloride). Any excess halogenting agent can be co-distilled with a higher boiling solvent under vacuum. The higher boiling solvents include, but are not limited to, aromatic hydrocarbon solvents, e.g., toluene, xylene and the like.

The reaction may be carried out at a temperature in the range of about 35° C. to about 100° C., and preferably at a temperature ranging from about 55° C. to about 75° C. The reaction between the imidazo[1,2-a]pyridine carboxylic acid and halogenating agent results in an intermediate acid halide, which is advantageously converted to the acetamide in situ by quenching the halide reaction mixture with an amine. The acetamide product precipitates out of the solution during quenching and can be separated by conventional techniques, e.g., filtering, washing and drying.

In step (b) of the process, the acid halide intermediate is reacted with the amine to form the compound of formula I. Preferably, the amine such as dimethylamine is added to the acid halide intermediate as an aqueous solution. The solution is preferably about 30 wt. % to about 50 wt. % amine, with the balance being water. If desired, the aqueous amine solution can be chilled to a temperature ranging from about −10° C. to about 10° C., and more preferably at a temperature ranging from about 0° C. to about 5° C. If desired, a quantity of the higher boiling solvent discussed above may be added and quenched into the chilled amine solution.

In another embodiment, the process of the present invention includes at least (a) reacting zolpidic acid with a halogenating agent in the absence of a solvent to form a zolpidic acid halide intermediate and (b) reacting the zolpidic acid halide intermediate with a dimethylamine to provide the free base of zolpidem.

In yet another embodiment, the process of the present invention includes at least (a) reacting zolpidic acid with a halogenating agent in the absence of a solvent to form a zolpidic acid halide intermediate and (b) reacting the zolpidic acid halide intermediate with a dimethylamine to provide the free base of zolpidem. This reaction is generally depicted below in Scheme I as follows:

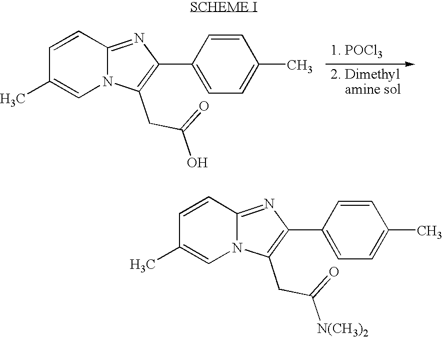

Next, the imidazo[1,2-a]pyridine derivatives can thereafter be converted to a pharmaceutically acceptable salt or hydrate, monohydrate, dihydrate, trihydrate, tetrahydrate and solvates thereof by methods known in the art. For example, the zolipem tartrate salt may be prepared by dissolving the free base of zolpidem in methanol and adding L(+)-tartric acid dissolved in methanol. The salt may then be crystallized from methanol.

The following examples are provided to enable one skilled in the art to practice the invention and are merely illustrative of the invention. The examples should not be read as limiting the scope of the invention as defined in the claims.

### EXAMPLE 1

Zolpidic acid (25 g) was suspended in neat phosphorus oxychloride (75 ml) and stirred under a nitrogen atmosphere. The reaction mixture was heated to a temperature ranging from about 60° C. to about 65° C. and stirred until the reaction mixture became clear. At this point in time, the reaction mixture was checked for the absence of zolpidic acid by TLC. Toluene (2×50 ml) was added to the reaction mixture and the phosphorous oxychloride was co-distilled with the toluene. Additional toluene (100 ml) was added and the reaction mixture was cooled to a temperature of about 50° C.

An aqueous solution of 40% dimethylamine (200 ml) was placed in a flask and cooled to a temperature ranging from about 0° C. to about 5° C. The reaction mixture containing the acid chloride was added slowly to the flask containing the chilled dimethylamine solution while the temperature was maintained at a range of from about 20° C. to about 25° C. The pH of the reaction mixture after the addition was about 9. The reaction mixture was stirred for 2 hours and the pH was checked. The solvent was distilled off completely under vacuum at a temperature below about 70° C. The reaction mixture, now in the form of a slurry, was cooled to a temperature ranging from about 0° C. to about 5° C. and stirred for 30 minutes. The product was filtered and washed with water to a neutral pH. The wet product was dried in an air oven at a temperature ranging from about 50° C. to about 55° C. until the moisture content was below 2%.

The dry product was suspended in ethyl acetate (3 volumes with respect to the dry product) and stirred at a temperature ranging from about 55° C. to about 60° C. for 1 hour. The product was then cooled to a temperature ranging from about 0° C. to about 5° C. and filtered. The wet cake was then washed with chilled ethyl acetate (2 volumes). The wet product was dried in an air oven at a temperature ranging from about 50° C. to about 60° C. until the moisture content was below 1%. Yield was 20 g, about 90% w/w, 71% theoretical yield. Purity: 99.8% by HPLC. ¹H NMR (DMSO, δ in ppm): 2.27 (s, 3H), 2.33 (s, 3H), 2.88 (s, 3H), 3.10 (s, 3H), 4.12 (s, 2H), 7.0-8.1 (aromatic, 7H). IR (KBr, cm⁻¹): 1635, 3423.

### EXAMPLE 2

Zolpidic acid (25 g) was suspended in chloroform (150 ml) and stirred under a nitrogen atmosphere. Phosphorus oxychloride (25 ml) was added to the suspension over a period of 5 to 10 minutes. The reaction mixture was heated to a temperature ranging from about 60° C. to about 65° C. and stirred for 6 hours. The reaction mixture became clear. The absence of zolpidic acid was checked by TLC. The reaction mixture was then cooled to a temperature of about 50° C.

An aqueous solution of 40% dimethylamine (200 ml) was placed in a flask and cooled to a temperature ranging from about 0° C. to about 5° C. The reaction mixture containing the acid chloride was added slowly to the flask containing the chilled dimethylamine solution while the temperature was maintained at a range of from about 20° C. to about 25° C. The pH of the reaction mixture after the addition was about 9. The reaction mixture was stirred for 2 hours and the pH was checked. The solvent was distilled off completely under vacuum at a temperature below about 70° C. The reaction mixture, now in the form of a slurry, was cooled to a temperature ranging from about 0° C. to about 5° C. and stirred for 30 minutes. The product was filtered and washed with water to a neutral pH. The wet product was dried in an air oven at a temperature ranging from about 50° C. to about 55° C. until the moisture content was below 2%.

The dry product was suspended in ethyl acetate (3 volumes with respect to the dry product) and stirred at a temperature ranging from about 55° C. to about 60° C. for 1 hour. The product was then cooled to a temperature ranging from about 0° C. to about 5° C. and filtered. The wet cake was then washed with chilled ethyl acetate (2 volumes). The wet product was dried in an air oven at a temperature ranging from about 50° C. to about 60° C. until the moisture content was below 1%. Yield was 23 g, about 90% w/w, 82% theoretical yield. Purity: 99.8% by HPLC. ¹H NMR (DMSO, δ in ppm): 2.27 (s, 3H), 2.33 (s, 3H), 2.88 (s, 3H), 3.10 (s, 3H), 4.12 (s, 2H), 7.0-8.1 (aromatic, 7H). IR (KBr, cm⁻¹): 1635, 3423.

### EXAMPLE 3

Zolpidic acid (25 g) was suspended in chlorobenzene (150 ml) and stirred under a nitrogen atmosphere. Phosphorus oxychloride (25 ml) was added to the suspension over a period of 5 to 10 minutes. The reaction mixture was heated to a temperature ranging from about 60° C. to about 65° C. and stirred for 6 hours. The reaction mixture became clear. The absence of zolpidic acid was checked by TLC. The reaction mixture was then cooled to a temperature of about 50° C.

An aqueous solution of 40% dimethylamine (200 ml) was placed in a flask and cooled to a temperature ranging from about 0° C. to about 5° C. The reaction mixture containing the acid chloride was added slowly to the flask containing the chilled dimethylamine solution while the temperature was maintained at a range of from about 20° C. to about 25° C. The pH of the reaction mixture after the addition was about 9. The reaction mixture was stirred for 2 hours and the pH was checked. The solvent was distilled off completely under vacuum at a temperature below about 70° C. The reaction mixture, now in the form of a slurry, was cooled to a temperature ranging from about 0° C. to about 5° C. and stirred for 30 minutes. The product was filtered and washed with water to a neutral pH. The wet product was dried in an air oven at a temperature ranging from about 50° C. to about 55° C. until the moisture content was below 2%.

The dry product was suspended in ethyl acetate (3 volumes with respect to the dry product) and stirred at a temperature ranging from about 55° C. to about 60° C. for 1 hour. The product was then cooled to a temperature ranging from about 0° C. to about 5° C. and filtered. The wet cake was then washed with chilled ethyl acetate (2 volumes). The wet product was dried in an air oven at a temperature ranging from about 50° C. to about 60° C. until the moisture content was below 1%. Yield was 20 g, about 90% w/w, 71% theoretical yield. Purity: 99.8% by HPLC. ¹H NMR (DMSO, δ in ppm): 2.27 (s, 3H), 2.33 (s, 3H), 2.88 (s, 3H), 3.10 (s, 3H), 4.12 (s, 2H), 7.0-8.1 (aromatic, 7H). IR (KBr, cm⁻¹): 1635, 3423.

### EXAMPLE 4

Zolpidic acid (25 g) was added to a 4-neck round bottom flask with ethylene dichloride (150 ml) and stirred under a nitrogen atmosphere. Phosphorus oxychloride (25 ml) was added to the suspension over a period of 5 to 10 minutes. The reaction mixture was heated to a temperature ranging from about 60° C. to about 65° C. and stirred for 6 hours. The reaction mixture became clear. The absence of zolpidic acid was checked by TLC. The reaction mixture was then cooled to a temperature of about 50° C.

The reaction mass was cooled to a temperature ranging from about 0° C. to about 5° C. Then the reaction mass was purged with dimethylamine gas slowly at a temperature ranging from about 20° C. to about 25° C. The pH of the reaction mixture was about 9. The reaction mixture was maintained at a temperature ranging from about 20° C. to about 25° C. for about 2 hours. The solvent was distilled off completely under vacuum at a temperature below about 70° C. The reaction slurry was cooled to a temperature ranging from about 0° C. to about 5° C. and stirred for 30 minutes. The solid was filtered and washed with water until there was a neutral pH. The solid was then dried at a temperature ranging from about 50° C. to about 55° C. until the moisture content was below about 2%.

The dry product was suspended in ethyl acetate (3 volumes with respect to the dry product) and stirred at a temperature ranging from about 55° C. to about 60° C. for 1 hour. The product was then cooled to a temperature ranging from about 0° C. to about 5° C. and filtered. The wet cake was then washed with chilled ethyl acetate (2 volumes). The wet product was dried in an air oven at a temperature ranging from about 50° C. to about 60° C. until the moisture content was below 1%. Yield was 23 g, about 90% w/w, 82% theoretical yield. Purity: 99.8% by HPLC. ¹H NMR (DMSO, δ in ppm): 2.27 (s, 3H), 2.33 (s, 3H), 2.88 (s, 3H), 3.10 (s, 3H), 4.12 (s, 2H), 7.0-8.1 (aromatic, 7H). IR (KBr, cm⁻¹): 1635, 3423.

### EXAMPLE 5

### Preparation of Zolpidem Tartrate

In a 4-neck round bottom flask, zolpidem (10 g) was dissolved in methanol (70 ml, moisture content 0.5%) and at room temperature (about 25° C. to about 30° C.) under stirring. Activated carbon (1.0 g) was added to the reaction mixture and stirred for 30 minutes. The reaction mixture was then filtered. The filtrate was taken in another round bottom flask and a solution of L-(+)-tartaric acid (2.44 g) prepared in methanol (24.0 ml) was added to the filtrate at room temperature. The reaction mixture was stirred for 2 hours at room temperature and then cooled to a temperature ranging from about 0° C. to about 5° C. The product was filtered and dried under vacuum at a temperature of about 40° C. Yield: 10 gm, about 100% w/w, 80% theoretical yield. Purity 99.85% by HPLC. [single impurity not more than 0.1%] ¹H NMR (D₂O, δ in ppm): 2.20 (s, 3H), 2.35 (s, 3H), 2.92 (s, 3H), 3.09 (s, 3H), 4.13 (s, 2H), 7.0-8.1 (aromatic, 7H). IR (KBr, cm⁻¹): 3542, 3456, 2921, 1635, 1513, 1405, 1200, 1125, 1072, 918, 854, 797, 683, 600. Mass-EI (m/z) M⁺ 307, 235, 219, 92.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of imidazo[1,2-a]pyridine derivatives of formula I: 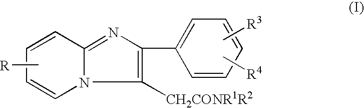
wherein R is hydrogen, halogen or a C₁-C₄ alkyl group; R¹ and R² are independently hydrogen, a straight or branched C₁-C₆ alkyl group which is unsubstituted or substituted by one or more halogen atoms, hydroxyl groups, N(C₁-C₄ alkyl)₂, carbamoyl or C₁-C₄ alkoxy radicals, a C₃-C₆ cycloalkyl radical, a benzyl radical, a phenyl radical or R¹ and R² together with the nitrogen atom to which they are bonded are joined together to form a substituted or unsubstituted heterocyclic group optionally containing one or more additional heterocyclic atoms; and R³ and R⁴ are independently hydrogen, halogen or a C₁-C₄ alkyl group, or a pharmaceutically acceptable salt thereof, the process comprising (a) reacting an imidazo[1,2-a]pyridine carboxylic acid of the formula II 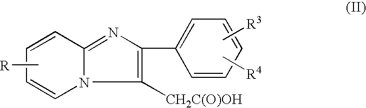
wherein R, R³ and R⁴ have the aforestated meanings with a halogenating agent in the absence of a solvent to form an acid halide intermediate and (b) reacting the acid halide intermediate with an amine of the formula HNR¹R² wherein R¹ and R² have the aforestated meanings to form the compound of formula I.

2. The process of claim 1, wherein R¹ and R² are each a straight or branched C₁-C₆ alkyl group.

3. The process of claim 1, wherein R¹ and R² are each methyl.

4. The process of claim 1, wherein R is a C₁-C₄ alkyl group, R³ is hydrogen and R⁴ is a C₁-C₄ alkyl group.

5. The process of claim 1, wherein the ratio of the imidazo[1,2-a]pyridine carboxylic acid of formula II to halogenating agent is about 1:2 w/v to about 1:3 w/v.

6. The process of claim 1, wherein the halogenating agent is a chlorinating agent.

7. The process of claim 1, wherein the halogenating agent is selcted from the group consisting of phosphorous oxychloride, phosphorous trichloride, phosphorous pentachloride and thionyl chloride.

8. The process of claim 1, wherein the imidazo[1,2-a]pyridine derivative of formula I is thereafter converted to a pharmaceutically acceptable salt or hydrate, monohydrate, dihydrate, trihydrate, tetrahydrate and solvates thereof.

9. A process for the preparation of zolpidem comprising:
(a) reacting zolpidic acid with a halogenating agent in the absence of a solvent to form an acid halide intermediate; and
(b) reacting the acid halide intermediate with dimethylamine to form zolpidem.

10. The process of claim 9, wherein the ratio of zolpidic acid to halogenating agent is about 1:2 w/v to about 1:3 w/v.

11. The process of claim 9, wherein the halogenating agent is a chlorinating agent.

12. The process of claim 9, wherein the halogenating agent is selcted from the group consisting of phosphorous oxychloride, phosphorous trichloride, phosphorous pentachloride and thionyl chloride.

13. The process of claim 9, wherein the zolpidem is thereafter converted to a pharmaceutically acceptable salt or hydrate, monohydrate, dihydrate, trihydrate, tetrahydrate and solvates thereof.

14. The process of claim 9, wherein the zolpidem is thereafter converted to a zolpidem tartrate salt.

15. A process for preparing zolpidem comprising
(a) reacting zolpidic acid with a halogenating agent in the absence of a solvent to form a zolpidic acid halide intermediate; and
(b) reacting the zolpidic acid halide intermediate with an aqueous solution of dimethylamine to form zolpidem.

16. The process of claim 15, wherein the halogenating agent is a chlorinating agent.

17. The process of claim 15, wherein the halogenating agent is selcted from the group consisting of phosphorous oxychloride, phosphorous trichloride, phosphorous pentachloride and thionyl chloride.

18. The process of claim 15, wherein the aqueous solution of dimethylamine comprises about 30 wt. % to about 50 wt. % dimethylamine.

19. The process of claim 15, wherein the zolpidem is thereafter converted to a pharmaceutically acceptable salt or hydrate, monohydrate, dihydrate, trihydrate, tetrahydrate and solvates thereof.

20. The process of claim 15, wherein the zolpidem is thereafter converted to a zolpidem tartrate salt.
